(19) United States
(12) Patent Application Publication
(10) Pub. No.: US 2008/0287873 A1
(43) Pub. Date: Nov. 20, 2008

(54) METHOD AND APPARATUS FOR CONTROLLING OPERATION OF A SYRINGE

(76) Inventors: Aldo LIBERATORE, London (CA); Mark Smith, London (CA)

Correspondence Address: BLAKE, CASSELS & GRAYDON LLP, BOX 25, COMMERCE COURT WEST, 199 BAY STREET, SUITE 2800, TORONTO, ON, M5L 1A9, CA

(21) Appl. No.: 12/102,234

(22) Filed: Apr. 14, 2008

Related U.S. Application Data
(60) Provisional application No. 60/911,589, filed on Apr. 13, 2007.

(51) Int. Cl. A61M 5/20

(52) U.S. Cl. 604/131

(57) ABSTRACT

A system is provided for controlling operation of a syringe. The system comprises a controller having an interface and at least one input mechanism; and a syringe gun communicably connected to the controller. The syringe gun comprises an elongated body with an open faced barrel cradle and open faced plunger bay for holding a syringe in the body, a plunger cradle moveable within the plunger bay for operating on a plunger of the syringe, and an actuating device for moving the plunger cradle according to a control signal. The controller generates the control signal for controlling the syringe gun.

This application claims priority from U.S. provisional application No. 60/911,589 filed on Apr. 13, 2007, the contents of which are incorporated herein by reference.

### TECHNICAL FIELD

The following relates to methods and apparatus for controlling operation of a syringe.

### BACKGROUND DESCRIPTION

A syringe is a well known device that is readily available and commonly used in a variety of medical applications. The syringe is typically used to inject a drug or other liquid into a patient either subcutaneously (i.e. under the skin) or intramuscularly (i.e. into the patient's muscle mass). The syringe may be pre-assembled with a predetermined quantity of content, or an administrator may be required to fill an empty syringe prior to use. It is also known that syringes can be used in other non-medical applications for administering quantities of a substance. Although the following is described in the context of medical applications, it will be appreciated that the following concerns and principles may also apply to such non-medical applications.

As part of an injection procedure, the administrator is sometimes required to choose the size (i.e. “gauge”) of the needle that is required to pierce the patient's skin in order to administer the contents of the syringe. Thinner (higher) gauges typically pierce the skin with greater ease thus causing less pain than thicker (lower) gauges. However, a thinner gauge can result in other consequential issues. Such issues include how the gauge of the needle affects the ability of the liquid to be injected or the amount of force required if the contents have a large viscosity. As shown in the chart in FIG. 9, the velocity of the liquid (or drug) as it exits the tip of the syringe needle increases substantially as the needle thickness is reduced (i.e. as gauge increases). As such, there are several factors that should be considered when choosing syringe needle parameters for a particular drug or application thereof.

The syringe can also be used to aspirate (i.e. withdraw fluids). For example, aspiration is commonly used by radiologists to withdraw fluids from glands for examination. Additionally, aspiration may be used to fill a syringe with a liquid or drug stored in a bulk container.

The above described procedures utilize some form of manual intervention. The consistency, accuracy and overall “quality” of an injection is dependent on the precision of the amount of fluid being injected or aspirated, as well as the relative control or “smoothness” of the rate of injection or aspiration. Several factors can impact an individual's effectiveness in administering a drug. A few examples of such factors include the amount of training given to the individual, the individual's experience, strength, fatigue and vision. Therefore, the variability of an injection between individuals and different injections from the same individual is naturally a concern in the medical industry.

Of late, evidence has been discussed indicating that the precision control of the rate of an injection can have many benefits such as: i) reduced muscular necrosis due to a slower rate of injection; ii) reduction in pain; iii) better absorption of the drug by the muscle mass; iv) potential for smaller doses; v) less stress being imparted on organs such as the liver; and vi) potentially higher effectiveness of the drug.

Additionally, there are drugs in development that depend on highly viscous gel like carriers to allow a slow release, thus extending the time between injections. As these carriers typically have a relatively high viscosity, they tend to require a thicker gauge needle and greater physical exertion to administer. As such, the needle gauge and force required by the individual can also be of concern.

It is therefore an object of the following to provide a method and apparatus to obviate or mitigate the above-identified disadvantages.

### SUMMARY

In one aspect, there is provided a syringe gun comprising an elongated body with an open faced barrel cradle and open faced plunger bay for holding a syringe in the body; a plunger cradle moveable within the plunger bay for operating on a plunger of the syringe; and an actuating device for moving the plunger cradle according to a control signal.

In another aspect, there is provided a system for controlling operation of a syringe comprising a controller comprising an interface and at least one input mechanism; and a syringe gun communicably connected to the controller, the syringe gun comprising an elongated body with an open faced barrel cradle and open faced plunger bay for holding a syringe in the body; a plunger cradle moveable within the plunger bay for operating on a plunger of the syringe; and an actuating device for moving the plunger cradle according to a control signal; wherein the controller generates the control signal for controlling the syringe gun.

In yet another aspect, there is provided a syringe gun comprising an elongated body with a plurality of barrels for containing one or more substances; a plurality of plunger bays for accommodating movement of a plurality of plungers operating with the plurality of barrel; a plurality of plunger cradles moveable within the plurality of plunger bays for operating on the plurality of plungers; and one or more actuating devices for moving the plurality of plunger cradles according to a control signal.

### BRIEF DESCRIPTION OF THE DRAWINGS

An embodiment of the invention will now be described by way of example only with reference to the appended drawings wherein:

FIG. 1 is a perspective view of a syringe control system.

FIG. 2 is a perspective view of a syringe gun holding a syringe and showing the underside thereof.

FIG. 3 is a perspective view of a syringe.

FIG. 4 is a sectional perspective view of a syringe gun showing a drive mechanism.

FIG. 5 is a plan view of the syringe gun shown in FIG. 4.

FIG. 6 is a sectional perspective view of a syringe gun showing a drive member extending from a drive mechanism.

FIG. 7 is a sectional perspective view of a syringe gun showing a plunger cradle and drive mechanism.

FIG. 8 is a perspective view of a syringe gun holding a syringe and showing the topside thereof.

FIG. 9 is a table showing fluid velocity versus needle thickness.

FIG. 10 is a schematic block diagram for the controller of FIG. 1.

FIG. 11 is a flow chart illustrating an initialization routine performed by the controller of FIG. 1.

FIG. 12 is a flow chart continuing from FIG. 11.

FIG. 13 is a flow chart continuing from FIG. 11.

FIG. 14 is an alternative flow chart for that shown in FIG. 13.

FIG. 15 is a perspective view of a syringe gun having a plurality of barrels.

### DETAILED DESCRIPTION OF THE DRAWINGS

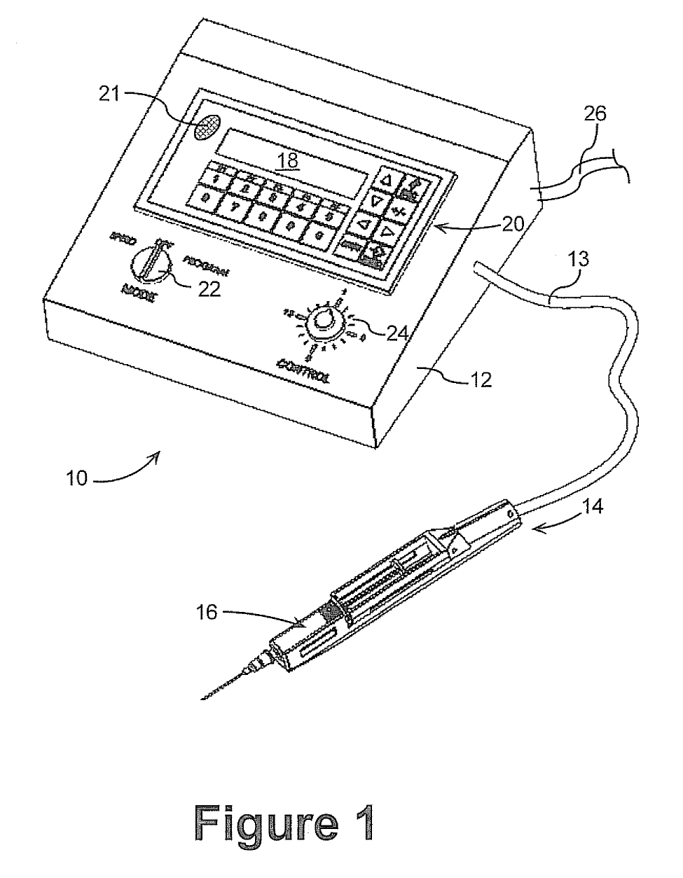

Referring now to FIG. 1, a syringe control system is generally denoted by numeral 10. The control system 10 comprises a controller console 12 and a syringe gun 14, connected to the console 12 via a connection 13. The syringe gun 14 holds a syringe 16 and controls operation thereof by receiving operating instructions from the console 12. The connection 13 is shown as being hardwired, however, it will be appreciated that the connection 13 may also be wireless.

The console 12 comprises a display 18 and a number of input mechanisms 20 such as functional keys as exemplified in FIG. 1 for entering parameters related to operation of the syringe gun 14. Optionally, the controller 12 may also include a speaker 21 for providing auditory feedback such as for an alarm. The console 12 also comprises a mode switch 24 for operating the syringe gun 14 and a control dial 24 for controlling injection volume rates applied by the syringe gun 14. In this example, the mode switch 24 includes three modes, namely an “OFF” mode to disable power to the syringe gun 14 to inhibit accidental use or to power down the syringe gun 14, a “Speed” mode to enable an operator to manually and immediately dial in a specified volume rate, and a “Program” mode to enable the operator to operate the syringe gun 14 according to a specific pre-programmed volume rate profile. The controller 12 may also comprise a communication link 26 for interfacing with an inventory tracking and documentation system such as that described in co-pending U.S. provisional patent application No. 60/889,670 filed on Feb. 13, 2007, the contents of which are incorporated herein by reference.

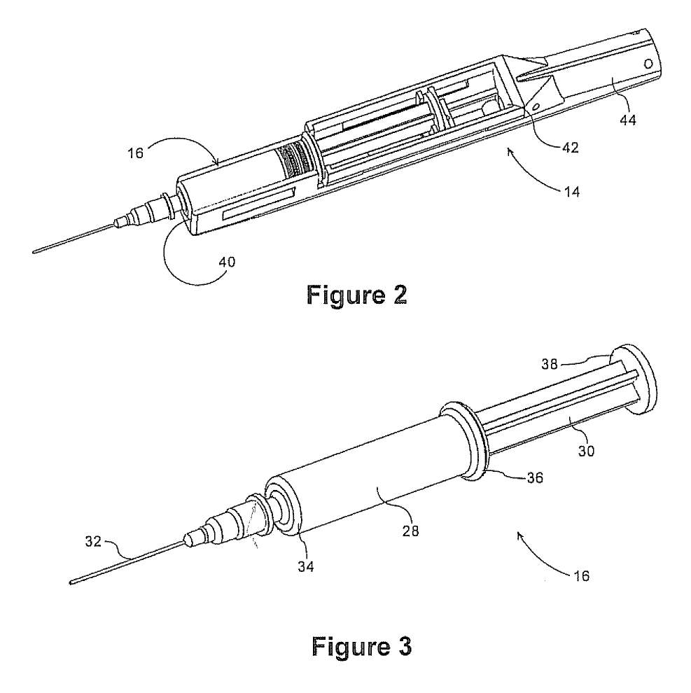

The syringe gun 14 loaded with a syringe 16 is shown in greater detail in FIG. 2 and the syringe 16 shown on its own in FIG. 3. The syringe gun 14 is configured to interchangeably and securely hold standard syringes, i.e. having the general characteristics shown in FIG. 3. Although there are many styles and sizes of syringes the majority of commercially available syringes 16 posses similar components.

FIG. 3 exemplifies a typical syringe used in the medical industry. The syringe 16 comprises a barrel 28, a plunger 30, and a needle 32. The needle 32 fluidly communicates with one end 34 of the barrel 28. The barrel 28 retains injectable fluid and guides the plunger 30. The needle 32 expels the contents of the barrel 28 as such contents are forced from the barrel 28 by the plunger 30. The plunger 30 is slidable within the barrel 28 between a barrel flange 36 and the one end 34 of the barrel 28. For manual operation of the syringe 16, the flange 36 is typically placed between the forefinger and the index finger to provide a grip. The plunger 30 is then used to push or pull a fluid through the needle 32 (i.e. for injection or aspiration respectively). The base of the plunger 30 itself has a plunger flange 38 which provides a surface for the thumb to bear against.

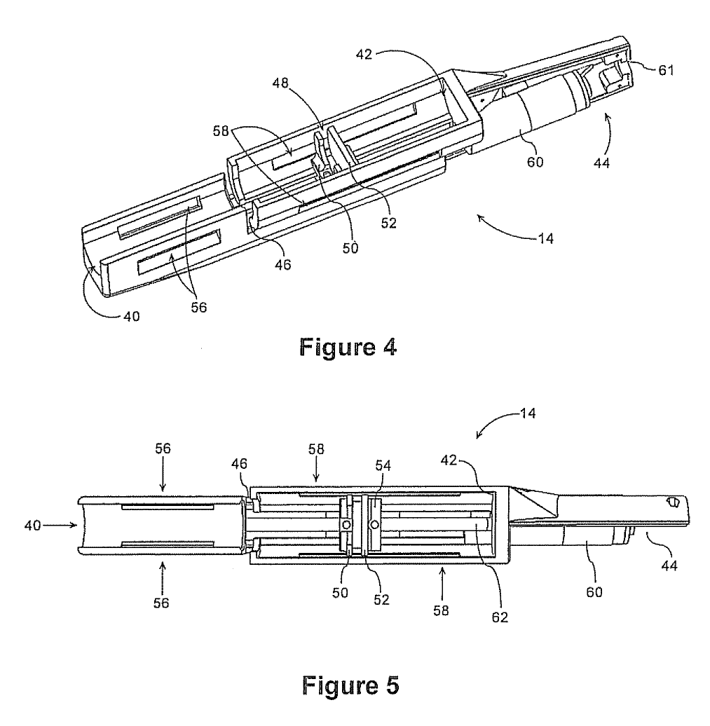

The syringe gun 14 is shown in greater detail in FIGS. 4 to 7. It can be seen that the syringe gun 14 has a slender profile to provide a “pen-like” feel for the user. The syringe gun 14 generally comprises a open faced barrel cradle 40 for securing the syringe barrel 28, an adjacent open faced plunger bay 42 to protect the plunger 30 from accidental external forces, and an enclosed linear actuator housing 44 adjacent the plunger bay 42 for containing a linear actuator 60 or similar drive mechanism to control movements of the plunger 30. The linear actuator 60 is preferably slender such that the actuator housing 44 is consistently sized with the plunger bay 42 and barrel cradle 40.

Situated between the barrel cradle 40 and plunger bay 42 is an integrated slot 46 for receiving the barrel flange 36. Extending from the linear actuator 60 along the bottom of the plunger bay 42 is a drive screw 62 having an attachment member 64 that translates along the drive screw 62. A slotted plunger cradle 48 is supported and attached to the attachment member 64 such that the plunger cradle 48 moves along the drive screw 62 with the attachment member 64. The plunger cradle 48 is defined by a forked portion 54 that is configured to accommodate the stem of the plunger 30 while limiting movement of the plunger flange 38 towards the barrel 28. The plunger cradle 48 also comprises an upstanding wall 52 spaced from the forked portion 54 such that the plunger flange 38 can be securely held between the forked portion 54 and the wall and to limit axial movement of the plunger 30. In this way, the plunger 30 can only be readily pushed or pulled under the control of the linear actuator 60.

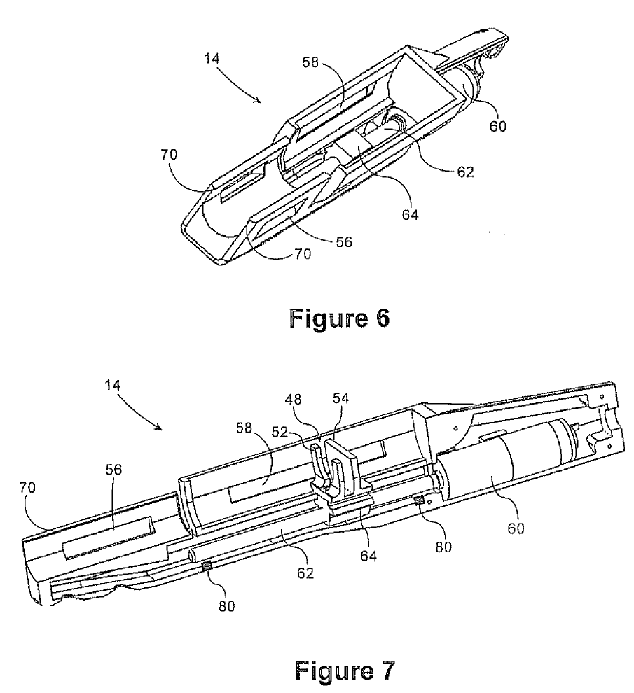

The integrated slot 46 and barrel cradle 40 are sized to be substantially similar or marginally smaller than the syringe barrel 28 and barrel flange 36 to allow the syringe 16 to be “snapped” into the syringe gun 14. To facilitate retention of the syringe barrel 28, a pair of opposing and protruding rails 70 extend along the length of the barrel cradle 40 as can best be seen in FIG. 6. As discussed above, syringes often vary m size and, as such, the plunger cradle 40, integrated slot 46 and plunger cradle 48 are preferably configured to adapt to a specific syringe size. However, the provision of the rails 70 and the inherent flexibility of the syringe gun 14 can be modified to accommodate a range of sizes. To achieve such flexibility, a material should be chosen for constructing the syringe gun 14 that can withstand repeated flexure imparted by the syringe barrel 28 and retain consistent pressure and holding forces with repeated use. There are many plastics and polymers available that can be considered that possess such qualities, such as nylons or glass reinforced nylons. The integrated slot 46 is configured to be similar in size or slightly larger than the barrel flange 36 such that even a small interference fit should be tolerated by the flexibility of the syringe gun material.

Alternatively, the barrel cradle 40 can be designed to have a maximum breadth based on typical syringe sized, wherein one or more shims or spacers or inserts can be inserted into the barrel cradle 40 to accommodate progressively smaller syringe sizes. In this way, an adjustable barrel cradle 40 can be provided. It will be appreciated that other adjustment mechanisms could also be used such as spring loaded side walls, barrel cradle cartridges etc.

The plunger cradle 48 should be capable of firmly secure the plunger flange 38 without allowing undue distortion during operation as it transfers forces from the linear actuator 60 to the plunger 30. By providing minimal surface area contact between the forked member 50 and the inner face of the plunger flange 38, distortion of the plunger flange 38 can be better accommodate during insertion.

To provide visual affirmation of the plunger 30 and contents of the syringe barrel 28, plunger view slots 58 and barrel view slots 56 respectively are provided. The barrel view slots 56 preferably extend along a substantial portion of the length of the side walls of the barrel cradle 40 as best shown in FIG. 4. Similarly, the plunger view slots 58 preferably extend along a substantial portion of the length of the side walls of the plunger bay 42. The view slots 56, 58 can be as wide as possible without compromising the structural integrity (e.g. stiffness) of the syringe gun 14. In general, the wider the view slots 56, 58, the more accommodating the view of the barrel 28 and plunger 30 as the syringe gun 14 rotates about its axis in use.

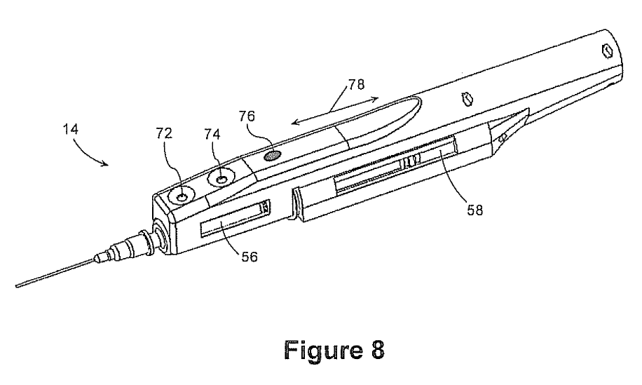

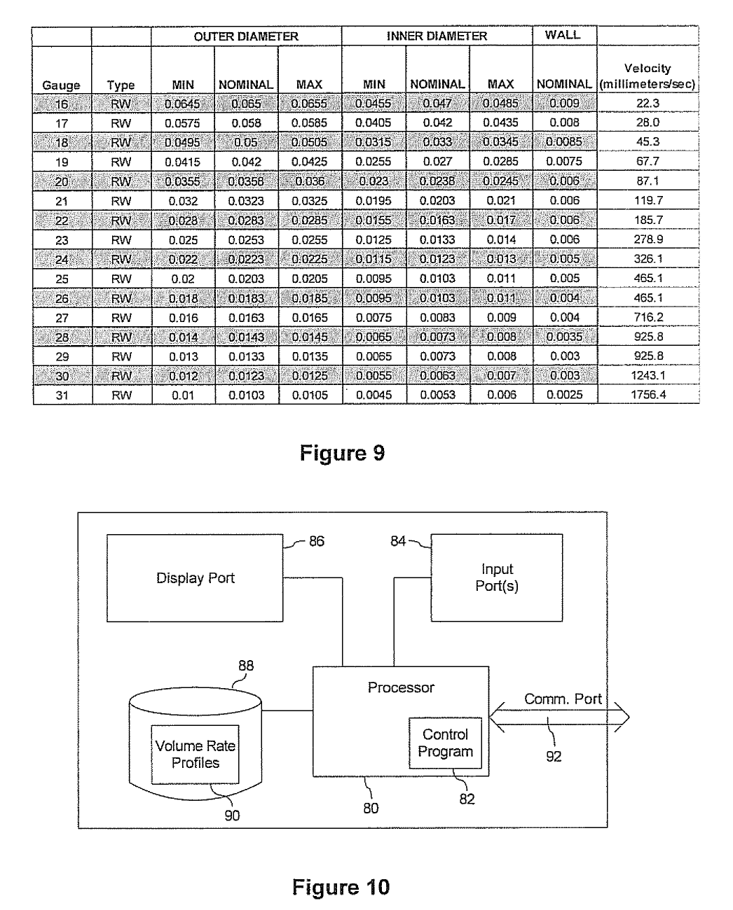

As can be seen in FIG. 8, a start push button 72 (i.e. for starting injection or aspiration sequence), a stop push button 74 (i.e. for ending sequence), and a light emitting diode (LED) 76 are provided along a profiled region 78 of reduced thickness on the face of the syringe gun 14 opposite the open face. The LED 76 provides a visual cue to the operator according to operation of the syringe gun 14. The start and stop buttons 72, 74 enable full control of the syringe gun 14 to enable the user to be in a comfortable and steady position before allowing the controller 12 to operate.

To secure the syringe 16 in the syringe gun 14, the plunger cradle 48 should be placed in an appropriate position. If the plunger 30 is at least partially extended (i.e. for injecting contents in the barrel 28), the plunger cradle 48 should be moved away from the integrated slot 46. If the plunger 30 is fully retracted (i.e. for aspirating fluids into an empty barrel 28), the plunger cradle 48 should be adjacent the integrated slot 46. The syringe 16 may then be placed atop the syringe gun 14 with the barrel 28 atop the barrel cradle 40 with the barrel flange 36 aligned with the integrated slot 46 and the plunger 30 along the plunger bay 42. Fine adjustments of the plunger cradle 48 can then be made to align the plunger cradle 48 with the plunger flange 38. Having this alignment, the syringe 16 may then be pressed into the syringe gun 14 preferably by applying force to the barrel 28 and plunger flange 38 simultaneously. The barrel cradle 40 should flex enough for the barrel 28 to “snap” into the barrel cradle 40 while the plunger flange 38 is secured firmly in the plunger cradle 48. In the result, the syringe gun 14 and syringe 16 assembly should appear as shown in FIG. 2. In order to properly align the plunger cradle 48 with the expected position for the plunger flange 38, the user can initiate a load function for aspiration or injection using the input buttons 20 on the controller 12.

Turning back to FIG. 8, the syringe gun 14 and syringe 16 assembly is sized and shaped to be comfortably held in one hand by the user, similar to the way in which someone would hold a pencil as discussed above. When being held by the user, the control buttons 72, 74 and LED 76 faced upwardly as seen in FIG. 8. The syringe gun 14 and syringe 16 assembly can rest in the nook of the thumb and forefinger while the end of the syringe gun 14 is held firmly between the thumb, index finger and forefinger. In this way, the forefinger has access to the control button 72, 74 allowing the user full control of the plunger 30. The placement of the view slots 56, 58 and the control buttons 72, 74 provides the necessary symmetry to enable ambidextrous operation.

To further aid the user, the LED 76 can be used in several ways to provide a visual cue based on the nature of the action. In one embodiment, the LED 76 is “on” whenever the plunger 30 is being moved, namely whenever an injection or aspiration is being performed. In another embodiment, the LED 76 may be multi-coloured such that one colour (e.g. red) is provided when the plunger 30 is injecting and another colour (e.g. green) is provided when the plunger 30 is aspirating. In yet another embodiment, the LED 76 can be configured to flash in sequence with the volume of fluid being injected or aspirated. For example, as the plunger 30 moves forward, the LED 76 is turned on until a prescribed amount of fluid is injected (e.g. 50 microlitres). As the plunger 30 continues to move forward, the LED 76 can then be either shut off(with a single colour LED) or the colour of the LED 76 changed (dual colour) to indicate that the prescribed amount has been injected. As the plunger 30 continues past the next prescribed amount (e.g. 100 microlitres), the LED 76 can be turned back on (or return to original colour).

Since the syringe 16 is securely supported in the syringe gun 14, movement of the plunger 30 is subject to the extent of movement of the linear actuator 60. To prevent jamming the plunger 30 or plunger cradle 48, the syringe gun 14 may be equipped with end-of-travel sensors 80 (see FIG. 7) at each end of the drive screw 62 to disable travel in the direction where damage would be imminent if allowed to continue. The end-of-travel sensors 80 may also be used to reset the linear actuator 60 by communicating with positioning software (not shown) designed into the linear actuator 60.

Turning back to FIG. 1, the controller 12 is designed to provide a user-friendly and easy to use device that can be understood by people with a variety of skills and training. The controls 20, 22, 24 and display 18 provide the user with an interactive console to enable such users to perform various injection and/or aspiration procedures consistently and accurately. Referring also to FIG. 10, the controller 12 comprises a processor 80, which stores a control program 82 for operating the controller 12 and syringe gun 14. The control program 82 contains computer readable instructions having computer code for causing the processor 80 to execute algorithms. The processor 80 communicates with one or more input ports 84 for receiving input from the input mechanisms, control dial 24 and mode control switch 22. The processor also communicates with a display port 86 for providing instructions to the display 18 for providing output for the user. The controller 12 also comprises a data storage device 88 such as a memory, which may be internal as shown or may also be external (not shown). The data storage device 88 is used to store volume rate profiles 90 that can be accessed by the processor 80 for inputting into the control program 82 to execute a particular algorithm. If applicable, the controller 12 also comprises a communication port 92 for interfacing with an external database via communication link 26. In this way, the profiles 90 can also be stored off-site and accessed by the controller 12.

Various injection profiles can be defined for various applications. For example, one injection profile can involve multiple injections of precise amounts of the same drug into the same patient (e.g. Botox® treatments). The profile would include the following steps: 1) Press start button; 2) Dwell for 5 seconds (allows operator to position needle without stressing skin or muscle); 3) Inject 0.1 ml at the rate of 0.01 ml/second; 4) Stop (wait for next start button signal); 5) Start button signal received; 6) Dwell 5 seconds; 7) Inject 0.12 ml at a rate of 0.01 ml/second; 8) Stop; and 9) Repeat cycle.

In another example, a profile can be defined for precision aspiration in radiology. Such a profile would include the following steps: 1) Press start button; 2) Dwell 2 seconds (allow operator time to steady the needle); 3) Aspirate 0.25 ml at a rate of 0.005 ml/second; and 4) Stop.

In yet another example, a profile can be defined for precision injection volume and rate with slight aspiration at end. Such a profile would include the following steps: 1) Press start button; 2) Dwell for 3 seconds; 3) Injection of 1.1 ml of drug at a slow rate of 0.005 ml/second; 4) Stop; 5) Aspirate 0.05 ml (allows withdrawal of needle from patient without spilling fluids on body); and Stop.

In general, the controller 12 operates according to user input, which is processed by the processor 80 by executing the control program 82, which may, if necessary, load a particular injection profile 90, e.g. in the program mode. It will be appreciated that the components shown in FIG. 10 are for illustrative purposes only and may be modified to support variations in options and features. For example, the display 18 may be a touch-sensitive screen which can also be used as an input mechanism. Also, the control program 82 may be stored il the data storage device 88 or in any other location so long as it can be stored, accessed and run by the processor. As such, any or all of the features shown in FIG. 10 can be implemented in hardware or software, e.g. as a program running from a personal computer or handheld device with a suitable connection to the syringe gun 14.

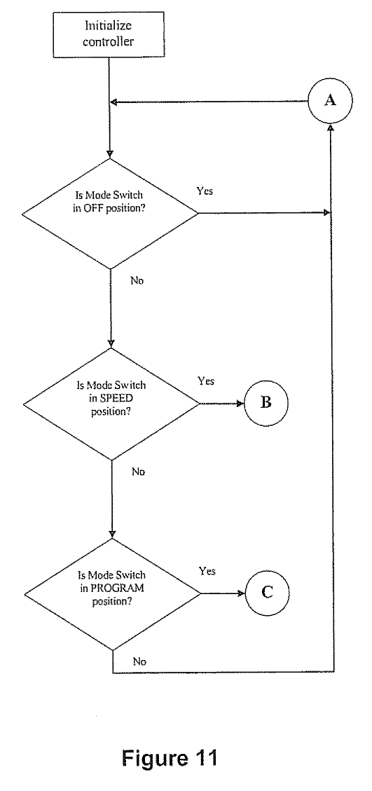

Operation of the syringe gun 14 using the controller 12 may be exemplified by referring to FIGS. 11 to 14. Referring first to FIG. 11, an initialization routine is shown. The controller 12 is first initialized, e.g. by selecting a power switch. The controller initialization may include a sub-routine for aligning the plunger cradle 48 according to the desired use (e.g. injection or aspiration) or this may alternatively be done after the controller 12 determines the desired use (e.g. according to an additional input or selection). The initialization also includes a check that a syringe 16 has been loaded. This can include reading a sensor (not shown) in the syringe gun 14 or by obtaining confirmation from the user that the syringe 16 has been loaded.

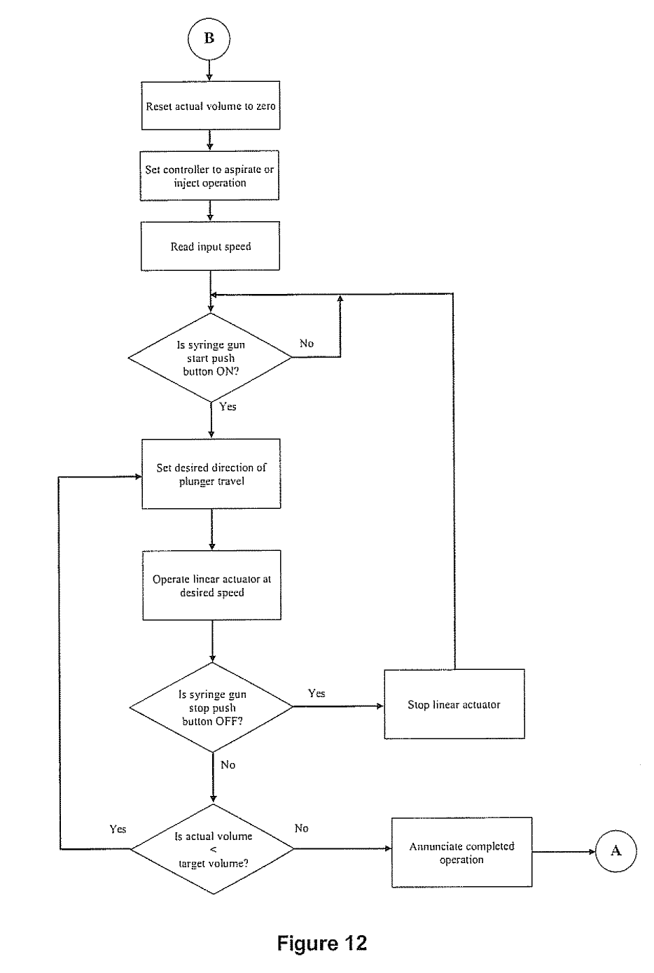

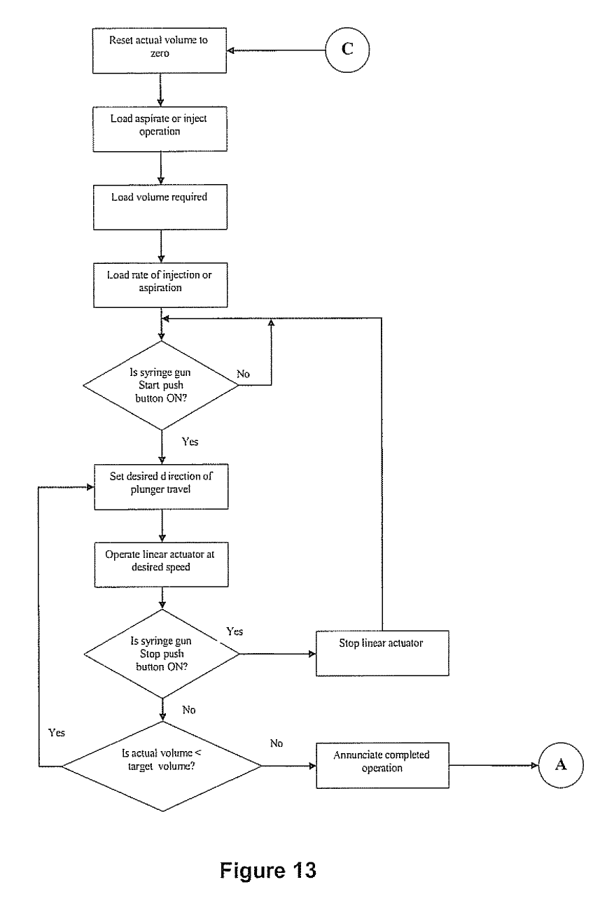

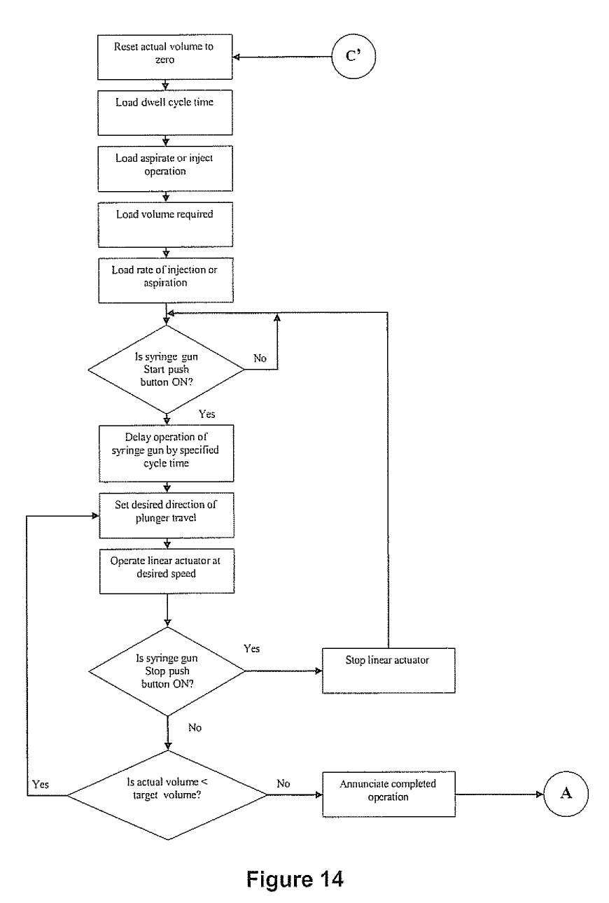

The controller 12 then steps through a series of checks to determine the selected mode of operation according to the selected mode control setting. If the mode control setting 22 is “Off” then the controller 12 idles until another setting is chosen. If the mode control setting 22 is “Speed” then the controller 12 proceeds to the routine shown in FIG. 12. If the mode control setting 22 is “Program” then the controller proceeds to either the routine shown in FIG. 13 or the routine shown in FIG. 14.

Turning now to FIG. 12, when the “Speed” mode has been selected, the user is able to dial in the desired volume rate using the speed dial 24. The controller 12 sets the actual volume to zero, which resets an internal value indicative of the current volume that has been aspirated or injected. If not already determined during initialization, the controller 12 may then prompt the user to select an inject or aspirate mode to determine the direction of travel for the plunger 30. The controller 12 also reads the speed dial 24 to determine the selected volume rate. Before the controller 12 can operate the syringe gun 14, the user should be in a ready position and select the start button 72. If the start button 72 has not been pressed, this indicates that the user is not prepared to begin and the controller 12 idles until it is determined that the user has pressed the start button 72.

The controller 12 may then begin the injection or aspiration process by first setting a direction of travel and then sending control signals to the linear actuator 60 to operate at the selected speed. The controller 12 continually checks that the stop button 74 has not been pressed and determines if the actual volume that has been dispensed or acquired is less than the target volume for the operation. The target volume may be set automatically according to the syringe 16 that has been loaded or can be entered by the user. Preferably, the controller 12 reads the target volume from an identifier such as a barcode or RFID tag. If the stop button 74 has not been pressed and the target volume has not been reached, then the plunger 30 continues to be pressed or pulled until either the stop button 74 is pressed or the target volume is met. If the stop button 74 has been pressed, this may indicate that the user wishes to pause, rest or otherwise cease the injection or aspiration. The controller 12 will then idle until the start button 72 is pressed, at which time it will determine if the remainder of the injection or aspiration needs to be performed. Once the target volume has been met, the controller 12 may then annunciate a completed operation to the user, either visually using display 18 or LED 76 or audibly using the speaker 21.

Control then returns to A shown in FIG. 11 where the controller 12 then determines if another operation is to be performed etc. When the mode switch is set to “Program” mode, the user is able to select a pre-programmed volume rate profile 90. In one embodiment, the controller 12 operates in “Program” mode as shown in FIG. 13. The controller 12 sets the actual volume to zero and then loads a selected profile 90 and determines whether the selected profile 90 is an aspiration operation or an injection operation. The profile 90 also indicates the volume of fluid in the syringe 16 that is required for the operation and sets this as the target volume. The profile 90 also indicates the rate of injection or aspiration, which is read by the controller 12.

Before the controller 12 can operate the syringe gun 14, the user should be in a ready position and select the start button 72. If the start button 72 has not been pressed, this indicates that the user is not prepared to begin and the controller 12 idles until it is determined that the user has pressed the start button 72.

The controller 12 may then begin the injection or aspiration process by first setting the direction of travel as read from the profile 90 and then sending control signals to the linear actuator 60 to operate at the speed indicated in the profile 90. The controller 12 continually checks that the stop button 74 has not been pressed and determines if the actual volume that has been dispensed or acquired is less than the target volume for the operation. If the stop button 74 has not been pressed and the target volume has not been reached, then the plunger 30 continues to be pressed or pulled until either the stop button 74 is pressed or the target volume is met. If the stop button 74 has been pressed, this may indicate that the user wishes to pause, rest or otherwise cease the injection or aspiration. The controller 12 will then idle until the start button 72 is pressed, at which time it will determine if the remainder of the injection or aspiration needs to be performed. Once the target volume has been met, the controller 12 may then annunciate a completed operation to the user.

Control then returns to A shown in FIG. 11, similar to completion of the Speed mode operation. In another embodiment, a delay may be incorporated into the injection and aspiration stages. Despite the increased control that the syringe gun 14 affords when compared to manual operation of a syringe 16, whenever a hand or finger is used to exert a force on the syringe gun 14 (e.g. by pressing the start button 72), there is a possibility that the force can alter the positioning or stability of the syringe gun 14 that results in an undesired movement of the tip of the syringe needle. To minimize such effects, the delay can be included in the volume rate profile 90 as an appropriate dwell time as noted in the above examples. This would allow, e.g., the user to press the start button 72 to initiate the injection or aspiration, and have enough tine to position the needle in the desired position until the delay has finished. Once the delay has finished, the injection or aspiration operation may commence. This enables the user to focus on steadying the position of the syringe gun 14 while still offering automatic control. FIG. 14 shows a modified version of FIG. 13 wherein the dwell time is loaded at the beginning of the routine and the delay effected after the start button 72 has been pressed.

It will be appreciated that other, more complex profiles 90 can be used. For example, different volume rates can be specified during corresponding stages in the operation. The controller 12 would then reference a timer in order to trigger a change in volume rate. Another example may involve a combination of both injection and aspiration stages where a first phase injects fluid at a certain rate then commences a second phase where a short aspiration is performed allowing the needle to be removed from the skin without dripping fluid on the patient.

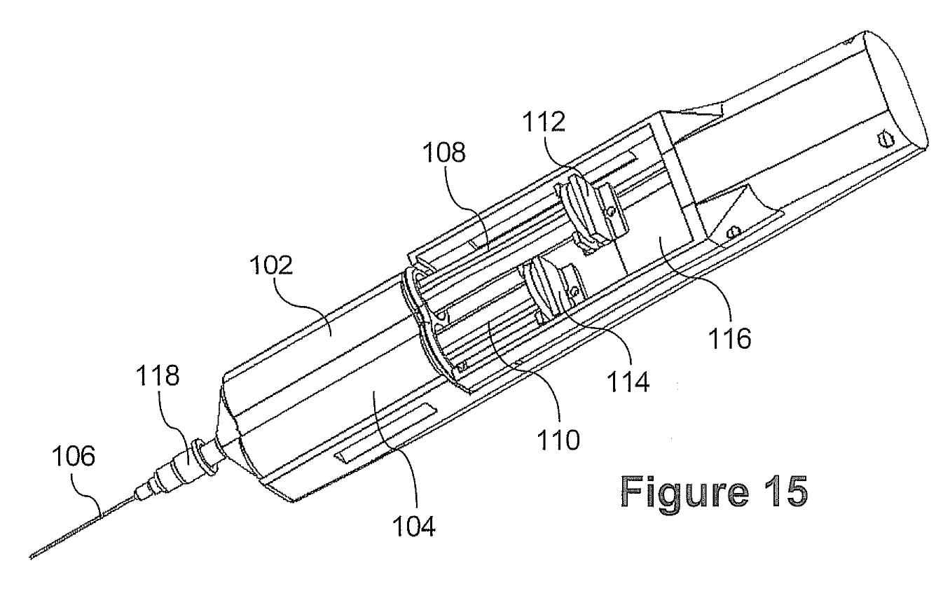

In another embodiment, a multi-barrel syringe gun 100 is provided as shown in FIG. 15. The multi-barrel syringe gun 100 is capable of independently controlling a plurality of syringe barrels 102, 104 that supply a single needle tip 106 as shown. A pair of respective plungers 108, 110 are operated by a pair of linear actuators (not shown in FIG. 15) acting, on a pair of plunger cradles 112, 114. As can be seen, the plunger bay 116 is sized to accommodate the additional plunger 110 and plunger cradle 114. It will be appreciated that the other components are similarly resized and proportioned accordingly. Operation of multiple barrels 102, 104 is preferably performed using a pre-programmed profile 90. In one application, two substances may be mixed prior to an injection. Another application is to aspirate with one barrel and inject with another. Yet another application is to supply two different fluids in a predetermined sequence according to a predetermined speed profile.

It will be appreciated that such operation would involve a profile that includes multiple stages that individually controls as many plungers as required for the particular step in the profile 90. For example, if two barrels (e.g. 102 and 104) are being simultaneously injected to mix the contents in the needle port 118, the linear actuators would be programmed to move the plunger cradles 112, 114 at specific rates and for specific amounts of time such that a specific quantity for each barrel enters the needle port 118 area for mixing. It will be appreciated that the actual rates, volumes and any delay times are entirely dependent on the profile 90, which is entirely dependent on the injection or aspiration sequence.

In yet another embodiment, the speaker 21 may be used as a buzzer having numerous modes that can signal an operation's status to the user. In one example, the buzzer may “chirp” 19 whenever a certain volume of fluid has been injected or aspirated. This could be used in both speed and program modes. In the program mode, the buzzer may be programmed to sound for an extended period (e.g. 1.5 seconds) to indicate a pre-programmed task has been completed. The buzzer may be used instead of or in conjunction with the LED 76 to provide numerous visual cues for the user to assist in the process.

Although the above has been described with reference to certain specific embodiments, various modifications thereof will be apparent to those skilled in the art without departing from the spirit and scope of the invention as outlined in the claims appended hereto.

What is claimed is:

1. A syringe gun comprising an elongated body with an open faced barrel cradle and open faced plunger bay for holding a syringe in said body; a plunger cradle moveable within said plunger bay for operation a plunger of said syringe; and an actuating device for moving said plunger cradle according to a control signal.

2. A system for controlling operation of a syringe comprising:
a controller comprising an interface and at least one input mechanism; and
a syringe gun communicably connected to said controller, said syringe gun comprising an elongated body with an open faced barrel cradle and open faced plunger bay for holding a syringe in said body; a plunger cradle moveable within said plunger bay for operating on a plunger of said syringe; and an actuating device for moving said plunger cradle according to a control signal;
wherein said controller generates said control signal for controlling said syringe gun.

3. A syringe gun comprising an elongated body with a plurality of barrels for containing one or more substances; a plurality of plunger bays for accommodating movement of a plurality of plungers operating with said plurality of barrel; a plurality of plunger cradles moveable within said plurality of plunger bays for operating on said plurality of plungers; and one or more actuating devices for moving said plurality of plunger cradles according to a control signal.
